(19) United States
(12) Patent Application Publication
(10) Pub. No.: US 2026/0117157 A1
(43) Pub. Date: Apr. 30, 2026

(54) VESSEL COMPONENTS FOR USE IN SMALL SCALE BIOREACTORS

(71) Applicant: Culture Biosciences, Inc., South San Francisco, CA (US)

(72) Inventors: Scot Fairchild, South San Francisco, CA (US); Collin Edington, South San Francisco, CA (US); Helen Luo, South San Francisco, CA (US); Jeffrey Thayer, South San Francisco, CA (US); Nicholas Lester, South San Francisco, CA (US); Jessica Lee, South San Francisco, CA (US)

(73) Assignee: Culture Biosciences, Inc., South San Francisco, CA (US)

(21) Appl. No.: 19/480,335

(22) PCT Filed: Apr. 25, 2025
(86) PCT No.: PCT/US2025/026523

§ 371 Date: Oct. 30, 2025

Related U.S. Application Data
(60) Provisional application No. 63/638,544, filed on Apr. 25, 2024.

(51) Int. Cl. C12M 1/00; C12M 1/06; C12M 1/12

(52) U.S. Cl. CPC C12M 23/40; C12M 27/02; C12M 29/08; C12M 29/20; C12M 37/02

(57) ABSTRACT

Bioreactors including bioreactors having features for improved performance, sensing and ease of use. Many embodiments provide bioreactors having improved dip-tubes, magnetically coupled agitators, condensers and sensing ability. Particular embodiments provide a bioreactor including a vessel having an inner volume for liquid contents and a head plate (HP) for coupling a plurality of components to the bioreactor where the HP is coupled to a top portion of the vessel and includes a plurality of ports. A diametric magnetic (DM) drive assembly (DMDA) and agitation shaft (AS) including at least one impeller are rotatably coupled to the HP. The DMDA and AS are rotated by non-vertical magnetic forces from a rotating DM positioned above the HP. A dip tube assembly (DTA) having a plurality of inner fluidic channels is positioned through a HP port such that a DTA end extends into the vessel for delivery liquids and sparging gasses.

### PRIORITY CLAIM

This application claims the benefit of and priority to U.S. patent application Ser. No. 19/189,101, filed on Apr. 24, 2025, and titled “Vessel Components for Use in Small Scale Bioreactors,” and U.S. Provisional Patent Application Ser. No. 63/638,544, filed on Apr. 25, 2024, and titled “Vessel Components for Use in Small Scale Bioreactors” both of which are incorporated herein for all purposes.

### BACKGROUND

A bioreactor refers to any manufactured device or system that supports a biologically active environment. In many applications a bioreactor comprises a specially designed vessel including nutrient inflow lines and sensors to support the growth of high concentrations of cells such as bacterial, mammalian or yeast cells.

Bioreactors are designed to consider various requirements to enhance their productivity for various products or applications. In some situations, bioreactors may be used in high throughput, automated fermentation systems that allow for controlled variations in the fermentation process. Small scale bioreactors, with a working volume ranging from 100-5000 mL, are typically used for high-throughput optimization of the growth parameters before scaling the process to larger reactors for validation and manufacturing. As such, it is desirable for these small systems to accurately replicate the larger production scale reactors, while being easy and economical to operate in large numbers. However, these design criteria present a number of technical challenges in particular one more or more of labor setup, efficiency in sampling and delivering liquids and gasses, ineffective condensers, accuracy of temperature sensing and durability of agitation systems which have yet to be addressed. Accordingly, there is a need for improved small-scale bioreactors.

### BRIEF DESCRIPTION OF THE DRAWINGS

The novel features are set forth with particularity in the appended claims. A better understanding of the features and advantages of the subject matter will be obtained by reference to the following detailed description that sets forth illustrative embodiments, in which the principles of the invention are utilized, and the accompanying drawings of which:

FIG. 1 is a perspective view of an embodiment of a bioreactor showing the bioreactor vessel, dip-tube, agitator, headplate and various bioreactor components coupled to the headplate;

FIG. 2 is a top view of an embodiment of a bioreactor headplate of FIG. 1 also illustrating components and connections to the headplate;

FIG. 3a is a side view of an embodiment of the bioreactor of FIG. 1;

FIG. 3b is a dimensioned side view of an embodiment of the bioreactor with values in millimeters;

FIG. 4 is a cross sectional view of an embodiment of a bioreactor multi-channel gas delivery manifold, showing one of the four sterile filters press-fit into the manifold tube;

FIG. 5a is a cross sectional view of an embodiment of a bioreactor having an agitator and dip tube assembly illustrating the dip tube assembly with multiple inner tubes extending out of the dip tube;

FIG. 5b is a cross sectional view of an embodiment of a bioreactor showing the agitator and a cut-away of the dip tube assembly revealing multiple tubes running to the dip tube plug;

FIG. 5c is a cross sectional enhanced view of the bottom of the bioreactor of

FIG. 5b, including a cut away view of the dip tube assembly illustrating positioning of the dip tube assembly and sparger within the vessel to position bubbles and fluid additions near high mixing zones of agitator impellers;

FIG. 6 is a perspective view of the top portion of an embodiment of the dip tube assembly including external tubing connections to the assembly;

FIG. 7 is a perspective view of the bottom portion of an embodiment of the dip tube assembly. ;

FIG. 8 is a cross sectional view of a bottom portion of an embodiment of the dip tube assembly illustrating the positioning of the dip tube plug to form an interference fit and fluidic seal with the outer dip tube and the plurality of inner tubes;

FIG. 9a is a cross-sectional view of the upper portion of an embodiment of the bioreactor including the headplate illustrating the wear-resistant bearing surfaces of the internal portion of the agitation system and the diametrically magnetized donut magnet positioned be coupled to a similar one (not shown) on the non-consumable portion of the gantry;

FIG. 9b is a close-up cross-sectional view of the agitation bearing system including the ball bearing, dowel pin and agitator shaft;

FIG. 9c is a cross sectional view of the upper portion of the bioreactor positioned below the gantry arm illustrating magnetic coupling of gantry magnet with the vessel magnet. where the north-south magnetic axis is left-right in the diametrically opposed magnets, rather than top-bottom;

FIG. 9d is an overhead view of the gantry including the gantry magnetic in place over headplate and magnetic drive assembly housing. The positioning of the chiller block (or other structure) adjacent to the condenser is also shown;

FIG. 10 is a cross sectional view of the bioreactor vessel and agitator shaft showing an embodiment of clip-on, modular impeller blades, and showing the cross-section of the thermal measurement well in the bottom right;

FIG. 11 is a cross-sectional close-up view showing an embodiment of the modular, clip-on impeller;

FIG. 12 is a cross-sectional view of the bioreactor illustrating an embodiment of the bioreactor condenser “chimney” configured to limit foam out, improve condensate capture, also illustrated are interfaces with the gantry chiller block;

FIG. 13 is a close-up cutaway view of the bottom of an embodiment of a bioreactor vessel having a thermal well for the positioning of a sensor; and

FIG. 14 is a dimensioned cross-sectional view of the bottom of an embodiment of a bioreactor vessel having thermal well illustrating the dimensions of the thermo-well with values in millimeters.

Note that the same numbers are used throughout the disclosure and figures to reference like components and features.

### DETAILED DESCRIPTION

Various embodiments provide improved bioreactors for growth of cells and other micro-organisms. Many embodiments provide single use bioreactors with features and subcomponents for improved performance including reductions in setup time, improved fluid delivery performance, and reductions in consumable cost. Particular embodiments provide improved bioreactor subcomponents including dip tube assemblies, headplates, condensers, magnetically driven agitator systems and vessels having structural features for more accurate temperature sensing.

By way of an overview, embodiments provide improved bioreactors. Many embodiments provide single use bioreactors with features and subcomponents for improved performance including reductions in setup time, improved fluid delivery performance, and reductions in consumable cost. Particular embodiments provide improved bioreactor subcomponents including dip tube assemblies, headplates, condensers, magnetically driven agitator systems and vessels having structural features for more accurate temperature sensing.

In a first aspect, embodiments provide a bioreactor for growing cells comprising a vessel defining an inner volume configured to contain culture media or other liquid contents, a head plate for coupling a plurality of components to the bioreactor and a dip tube assembly. The head plate is coupled to a top portion of the vessel and includes at least one port. The dip tube assembly is positioned within one of the ports such that a top portion of the dip tube extends above and out of the headplate and a bottom end of the dip tube extends into the vessel inner volume.

The dip tube assembly comprises an outer tube having a top end, a bottom end and a side wall defining an interior volume. In some instances, the dip tube assembly may be referred to as a dip tube or DTA. A plurality of inner tubes for delivery and/or sampling of liquids and gases are disposed within the interior volume of the outer tube. Each inner tube includes a top and bottom end and lumens for the passages of liquids and gases. In one or more embodiments the DTA can include between four to ten inner tubes which may be evenly radially distributed within the interior volume or in some instances one inner tube will be positioned at a center of the outer tube and the other inner tubes distributed around it. In particular embodiments, the DTA will include seven inner tubes, one of which is positioned at the center of the outer tube and the other six radially distributed around the center. The center positioned inner tube will typically be used for delivery of sparging gas.

A plug is positioned within a bottom portion of the interior volume of the outer tube and will typically have a rounded shape to fit into the interior volume of the outer tube. The plug includes a plurality of lumens in/through which the plurality of inner tubes is positioned. In some embodiments, the bottom ends of the inner tubes extend out of the bottom surface of the plug by a selected amount for example in a range of 1 to 5 mm or 2 to 3 mm. In other embodiments, the bottom ends of the inner tube are substantially flush with the bottom surface of the plug.

The shape, material properties and other features of the plug are desirably configured to form a fluidic seal around each inner tube such that fluid contents of the bioreactor do not enter the interior volume of the outer tube when the dip tube assembly is positioned within the bioreactor. The plug also includes at least one protrusion positioned on a side surface of the plug. The protrusion(s) is sized to form an interference fit and fluidic seal between the plug and an interior surface of the outer tube such that liquid contents of the bioreactor do not enter the interior volume of the outer tube when the dip tube assembly is positioned within the bioreactor. Typically, the plug will include two such protrusions and they will extend around the entire circumference of the plug. In one or more embodiments, the plug has a coefficient of thermal expansion matched to a coefficient of thermal expansion of the outer tube such that the fluidic seal between plug and the outer tube is maintained upon heating of the outer tube by culture media or other liquid contents of the bioreactor.

In many embodiments the DTA will also include a down tube for the delivery of sparging gas to a selected location in the bioreactor vessel. The downtube (also sometimes referred to herein as a downpipe sparger) is coupled to a bottom portion of the plug and includes an inner lumen that is fluidically coupled to a bottom end of one of the inner tubes. Typically, the down tube which will be coupled to a center portion of the plug bottom surface and as such will be fluidically coupled to be a center positioned inner tube as described above. However, other locations for positioning of the down tube on the plug surface are also contemplated. In many embodiments, the down tube will include an elbow portion that is shaped or otherwise configured to direct a bottom end of the down tube (including the down tube inner lumen) near or towards a high mixing zone of an agitation impeller or other agitation element coupled to an agitation shaft within the bioreactor vessel. In some embodiments the down tube and elbow portion can be configured (e.g., sized and shaped) to locate the down tube end within 5 to 20 mm from an agitation impeller. In particular embodiments, the down tube and elbow portion can be configured to position the dip tube end at a location between impeller and the bottom of the bioreactor vessel (e.g., equidistant between the two). In various embodiments, the exact location of the down tube end can be selected depending upon one or more of the volume of liquid in the vessel, the rotational velocity of the agitation shaft and the flow rate of the sparging gas as well as the desired dissolved gas concentration (e.g., O2) to be obtained within the culture media or other vessel liquid. In some embodiments, selection of the down tube end position can be achieved by embodiments of the downtube which are configured to be telescoping and/or through the use of a set of detachable down tubes of various lengths.

In use, embodiments of the bioreactor having a dip tube assembly including those with a down tube provide the advantage of aiding to repeatably position all the gas/liquid addition and sampling ports at a defined location and in the gas of sparging gas near to the agitation impeller(s) to ensure fast mixing and in turn reproducibility of results across reactors.

In some embodiments, the bioreactor will also include a multiport gas manifold (MPGM) which includes a plurality of separate gas channels each having an inlet and outlet and conduit extending from the channel outlet. Typically, the channels will have a tapered shape with the inlet being larger than the outlet. At least one of the channels is fluidically coupled to the dip tube assembly for the delivery of a sparging gas through the assembly. One of the channels can also be coupled to the headplate for the delivery of gas (known as overlay gas) to the space in the vessel above the liquid contents (e.g., the culture media). In particular embodiments, the MPGM includes four gas channels, two coupled to the dip tube for the delivery of sparging gas (e.g., O2, CO2, Nitrogen, etc.), one coupled to the headplate for the delivery of overlay gas and one for gas leaving the vessel described herein as off-gas which will typically go through a condenser such as that described herein.

In various embodiments, the MPGM can have various features and attributes to facilitate connections at the channel inlet and outlet, control gas flow rates and maintain sterility of gases flowing into the bioreactor.

Also in some embodiments, the channels of the MPGM may contain or be configured to contain sterile filters for filtering out microbes (e.g., bacteria, fungi and viruses) and particulates. The filters may be a standard shape or custom fitted for the shape of the gas channels. Typically, they will be press fit into the gas channel. In particular embodiments, the filters may have a pore size of 0.2 μm or less. Also, in some embodiments the channel outlets can include an extended fitting to allow for insertion and secure connection of tubing or other conduit connecting the outlets to the DTA or headplate. Also, the channel inlets can include or be configured to be coupled with rigid O-rings seals (e.g. fabricated from high durometer silicone) which serve to ensure that reliable connections are made to all of the gas channel inlets. Additionally in one or more embodiments, the MPGM inlets (or other portion of the channels) may include or be configured to be coupled to one or more mass flow controllers so as to control the flow of gas into each channel.

In various embodiments, the headplate may also include various features in addition to the dip tube assembly to facilitate the delivery of liquids to the dip tube and bioreactor. For example, in one or more embodiments, the headplate or other portion of the bioreactor is coupled or is configured to be coupled to a multi-port fluid (e.g. liquid) manifold (MPFM) including a plurality of separate channels for fluidically coupling at least a portion of the inner tubes to separate sources of liquid, such as liquid filled syringes. Typically, the MPFM will include five ports and corresponding fluid channels, but other numbers are also contemplated. The ports of the MPFM can be coupled to external fluid segments which are supplied with the bioreactor and configured to be coupled to syringes or pumping/fluid source means which are used to deliver fluid to the DTA and/or headplate.

In still other embodiments, the headplate includes a drip feature which may be molded into the headplate or positioned in one of the ports for drip delivery of fluids such as an antifoaming agent to the culture media or other liquid contents of the vessel. In particular embodiments, the drip feature is positioned at the center of the headplate so as to have drops delivered to the center of the vessel.

Also in various embodiments, the one or more ports in the head plate may also include an expansion port, or multiple ports, that can be configured and used to add an additional probe or related component such as redundant glass pH probe, redundant oxygen probe, or other standard-sized threaded probe such as those for cell density, Raman spectroscopy, glucose/lactate, or other Process Analytical Technology probes. Typically, the expansion port will include a removable cover allowing a user to easily open the port as needed to add the additional component from within a sterile environment.

In another aspect, embodiments provide a bioreactor for growing cells comprising a vessel defining an inner volume configured to contain culture media or other liquid contents and a head plate for coupling a plurality of components to the bioreactor wherein the headplate includes a condenser. The head plate is coupled to a top portion of the vessel and includes at least one port and a condenser for condensing water vapor from gas flowing out of the bioreactor through the condenser. The condenser has a vertical oblong exterior shape rising above the headplate defining an interior volume, an oblong horizontal shape including two long sides and two short sides and a bottom portion and a top portion. The bottom portion has an oblong opening continuous with the headplate and the top portion includes an opening positioned at a short side for the outflow of gas. At least one of the long sides is configured to be thermally coupled with a movable chilling structure such as rectangular plate. Also at least one of the interior condenser shapes or horizontal dimensions along a vertical axis of the condenser are configured to minimize blockage of the interior volume by liquid or foam bridging or otherwise spanning across interior walls of the condenser.

The horizontal profile or cross-section of the condenser including the bottom portion and bottom opening along with the top portion will typically have an oval or other oblong asymmetric shape sized to inhibit or reduce liquid or foam blocking the condenser bridging or otherwise spanning across the interior walls of the condenser. Accordingly, in these and related embodiments the long sides of the condenser will be flat and the short sides curved. The condenser will also typically have a decreasing vertical taper to facilitate condensation of water vapor and provide for ease of outflow gas connection to the condenser.

In one or more embodiments, at least one of the shape or dimensions of the condenser are configured to minimize loss of vessel liquid content resulting from condensed liquid escaping out of the gas outflow opening and/or inefficient condensation of gas flowing through the condenser. Accordingly, in these and related embodiments the condenser long sides can have a length in a range from about 20 to 30 mms, the short sides a length in a range from about 5 to 10 mm and the height of the condenser can range from about 50 to 70 mm. In these and related embodiments the liquid loss can be less than about 5 percent of the vessel liquid contents per day of operation of the bioreactor and more preferably 1 about per cent per day of operation of the bioreactor. Also, in these and related embodiments, the condenser shape and dimensions provide for sufficient internal surface area to condense at least about 90 per cent of the water vapor flow through the condenser.

Various embodiments of the condenser can also include other features and aspects to reduce or prevent blockage of the interior space of the condenser by water droplets and/or foam. For example, in some embodiments, an interior surface of the condenser has a hydrophobic surface tension configured to minimize condensed liquid from adhering to the interior surface. Desirably, the interior surface tension is configured to induce condensed liquid to fall or roll down the condenser interior surface. In one or more embodiments, the surface tension of the condenser can be below about 50 dynes/cm, more preferably below 40 dynes/cm and still more preferably below about 30 dynes/cm.

Various embodiments of the condenser can also be configured to enhance cooling of the condenser by the chilling structure and thus condensation of water vapor flowing through the condenser. For example, in one or more embodiments the long side(s) for thermal coupling to the chilling structure can include an additive or coating for enhanced thermal conductivity. Such coatings or additives can include thermally conductive polymers or metals known in the art. In these and related embodiments including thermally conductive coatings or additives, the thermal conductivity of the thermally coupled long side can be at least about 1 W/(m K) or greater and more preferably at least about 10 W/(m K) Additionally in one or more embodiments, the condenser structure including the long side(s) for thermal coupling can be configured to be put under compressive loading or force by the chilling structure (when it is moved into place against the long side e.g., by movement of the gantry) so as to enhance conduction and heat flux between from the condenser structure including the long side to the chilling structure. In these and related embodiments, the surface contour of the long side can substantially match or correspond to that of the chilling structure. For example, for embodiments of a rectangular shaped chilling structure the contour of the thermally coupled long side can be substantially flat. Similarly, for embodiments where the chilling structure has curved shape the contour of the coupled long side can have a matching curved shape.

In still another aspect, embodiments provide a bioreactor for growing cells comprising a head plate and a coupled vessel defining an inner volume to contain liquid contents wherein the vessel has a structure for enhanced accuracy of temperature measure by a thermal probe. The head plate is coupled to a top portion of the vessel and includes one or more ports or other means for coupling various components to the bioreactor. The vessel includes side and bottom walls with the latter having a thermal well comprising an upwardly extending cavity for insertion of a thermal probe to measure the temperature of the liquid contents of the vessel. Desirably, the cavity shape, height and wall thickness are configured to allow for greater than a 99 to 99.5 percent accuracy in a temperature measurement of liquid contents surrounding the cavity by the inserted thermal probe. Typically, the cavity will have an upward cylindrical shape with a curved end and also may have a decreasing vertical taper.

In one or more embodiments, the dimensions of the cavity can be configured for a slightly snug fit around the temperature probe such that surface of the temperature probe makes complete or near complete contact with the surface of the cavity (i.e., the surface of the vessel bottom wall defining the cavity) for enhanced thermal conduction the cavity wall to the temperature probe. Accordingly, in these and related embodiments the diameter and length of the cavity can be selected to match those of various temperature probes known in the art including specific temperature probes used for measurement of bioreactor temperature. In particular embodiments, the cavity has a height between about 13 to 15 mms and a width of between about 3 and 3.4 mm and a wall thickness between about 0.4 mm and 0.6 mm with a specific embodiment of about 14 mm height, a 3.2 mm width and a 0.5 mm wall thickness.

In addition to dimensions, one or more embodiments of the thermal well may include other means for improving accuracy of temperature measurements by the inserted temperature probe. For example, similar to the thermally coupled condenser long side, in some embodiments, the outer walls of the thermal well or cavity may include a coating or additive for enhanced thermal conductivity.

In still another aspect, embodiments provide a bioreactor for growing cells comprising a vessel defining an inner volume configured to contain culture media or other liquid contents, a head plate for coupling a plurality of components to the bioreactor, and an agitation assembly rotatably coupled to the headplate. The head plate is coupled to a top portion of the vessel and includes more ports or other means for coupling various components to the bioreactor. The agitation assembly comprises a magnetic drive assembly, an agitation shaft coupled to the drive assembly, and at least one impeller coupled to the agitation shaft. The magnetic drive assembly includes a protective housing and a first diametric magnet positioned in the housing and configured to be magnetically coupled to a second diametric magnet positioned above the headplate in a rotating housing so as to rotatably drive the agitation shaft by rotation of the second magnet. The second diametric magnet rotatably drives the first magnet by magnetic lines of force substantially orthogonal to an axis of rotation of the two magnets. In many embodiments, the first and second diametric magnets have a toroidal shape such as a square, rectangular or circular toroid; however other shapes for the diametric magnets are also contemplated.

In various embodiments, the drive assembly housing comprises a first part and a second part which is fixedly inserted into the first part to define an interior space containing the first magnet and form a substantially watertight seal around the interior space and the magnet. The second part is fixedly attached to a proximal end of the agitation shaft. In particular embodiments, the first and second housing parts can be configured for the second part to have a snap fit into the first part using protrusions and/or detent features in one or both parts.

In embodiments, the magnetic drive system housing also includes a bearing system for reducing friction during rotation of the agitation shaft. In particular embodiments, the bearing system can be at least partially positioned or contained in a recess formed in the first housing part. According to various embodiments the bearing system comprises a bearing, a first bearing contact structure positioned below the bearing and a second bearing contact structure positioned above the bearing. The first bearing structure will typically comprise an elongated stainless steel dowel pin or other metal pin that is fixed and inserted into the recess. Such elongated metal dowel pins or other like structures provide the benefit of conducting heat away from the bearing surface, reducing wear of the bearing. The second bearing contact structure may comprise a post or other structure that is fixedly inserted into a surface of the headplate, typically at the center of the headplate. The bearing will typically correspond to a ball bearing but configurations using roller bearing or even magnetic bearings are also contemplated. For embodiments of the bearing system using ball bearings, the bearing may comprise one or more wear resistant materials known in the art including for example various wear resistant polymer such as polyamide-imide with a specific example being TORLON. Also, for embodiments where the bearing is a ball bearing, the post or other second bearing contact structure can have a cup-shaped contact surface configured to center the bearing. In specific embodiments, the radius of curvature of the cup surface can correspond to that of the ball bearing.

In such embodiments of the magnetic drive and bearing system as described above, the systems are configured to operate such that during rotational movement induced by the second magnet, the first magnet rotates (along with the agitator shaft and the dowel pin) while the ball bearing remains stationary, forming the wear surface at the intersection of the dowel pin and ball bearing. The metal dowel pin also provides an additional function and benefit of drawing heat down and away from the wear surface to increase lifetime of the bearing system and prevent overheating.

In various embodiments, the second diametric magnet will typically be positioned in a rotatable housing positioned in proximity to the headplate outer surface above the drive system housing such that the two diametric magnets are substantially axially aligned. In many embodiments the rotating housing is positioned or otherwise coupled to a movable gantry configured to move the rotatable housing and second magnet in axial alignment with the drive housing and first magnetic so as to magnetically couple the two magnets.

In some embodiments of the magnetic drive system, the bioreactor headplate can include a raised portion and the magnetic drive system housing can be at least partially positioned within the raised portion. Such embodiments provide the advantage of reducing the space requirements for the drive system housing and position the housing away from the liquid in the vessel reducing the likelihood of vessel liquid contents from getting on or into the housing. They also facilitate positioning. alignment and magnetic coupling of the rotatable housing/second magnetic with the drive housing/first magnet as owing to the height of the raised portion above the headplate surface (which can be in the range of 10 to 30 mm) two housing can be brought into close proximity without interference by other components of the headplate.

In addition to the benefits described above, embodiments of the bioreactors and subcomponents also provide improved flexibility to accommodate various applications including for example incubation and growth of cell populations used for the production of biologics and cell therapy products as well as production of viral vectors. In particular, embodiments of the bioreactor described may provide improved modularity with one or more components of the bioreactors capable of being individually modified or customized to meet the cell incubation and growth requirements at a fine-tuned level. Embodiments of a single use bioreactor including those of the bioreactor vessel can be scaled to any suitable size for example from 250 to 5000 ml with specific embodiments of 300, 500, 1000, 1500, 2000, 2500, 3000 and 4000 ml. Embodiments of the single use bioreactors and subcomponents of the presentation may also be designed and configured to operate under control of an automated system including for example a cloud based remotely operated system for performing a design of experiments on optimal conditions for cell growth for one or more applications.

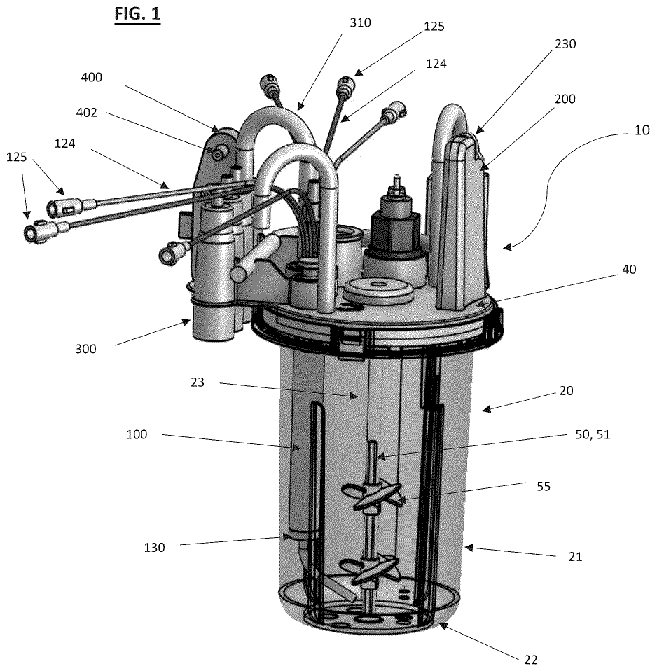

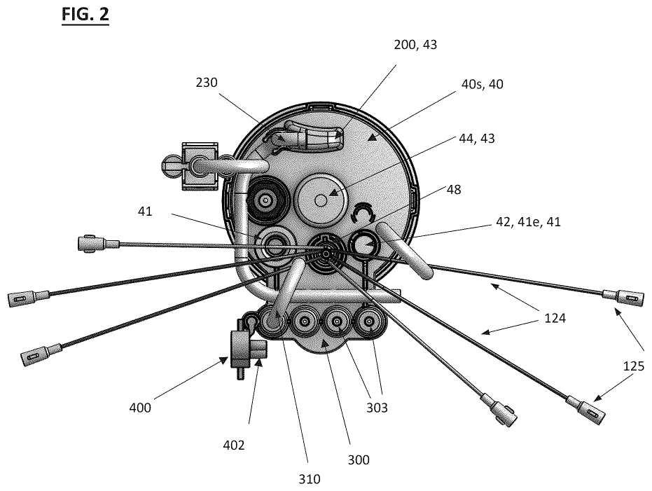

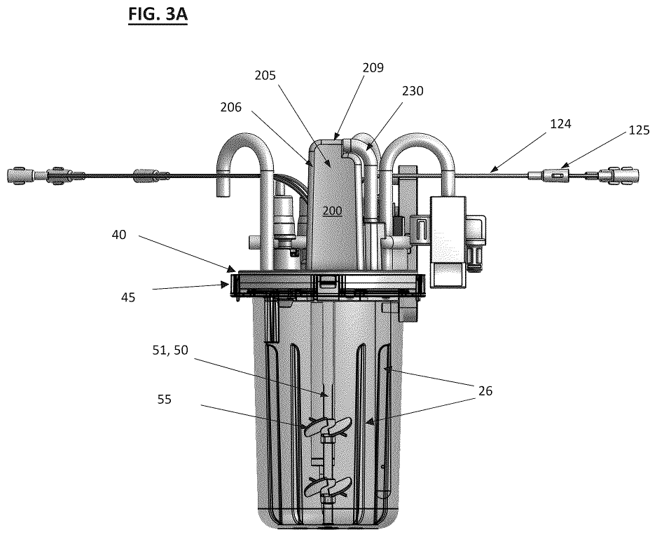

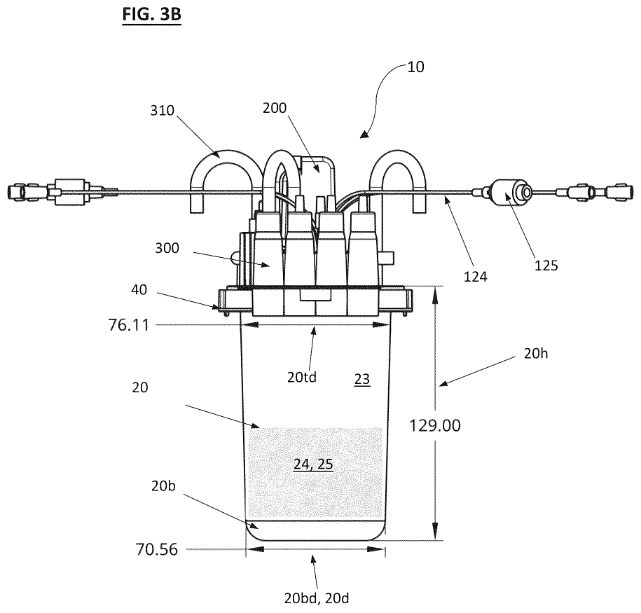

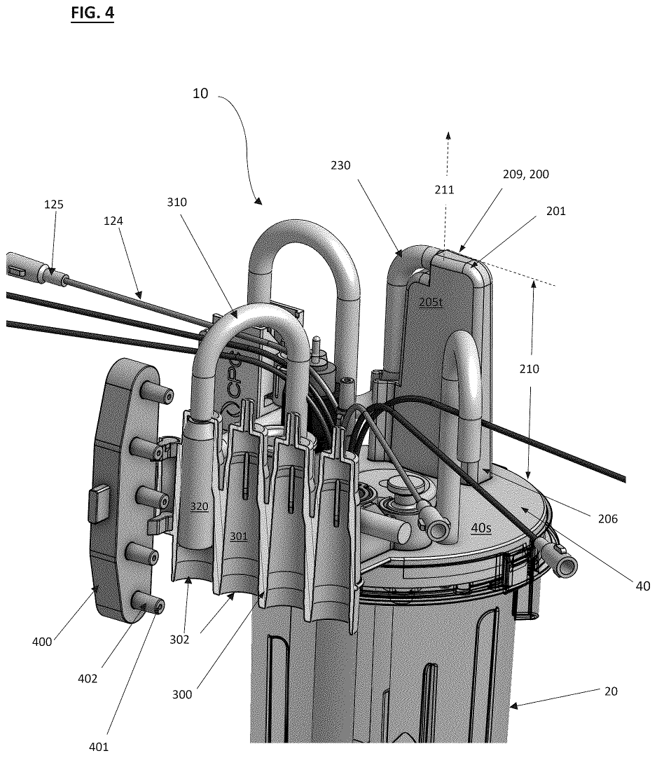

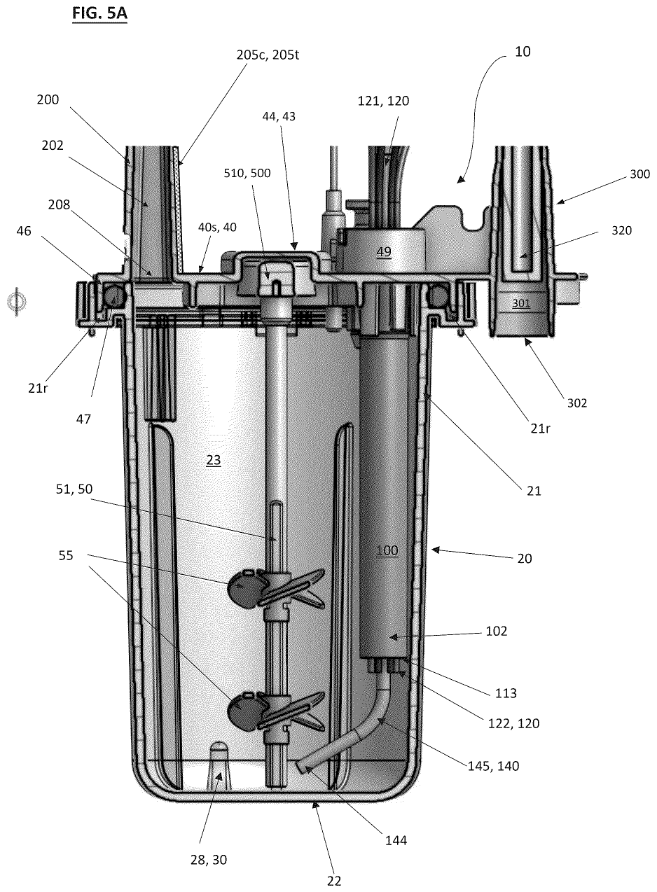

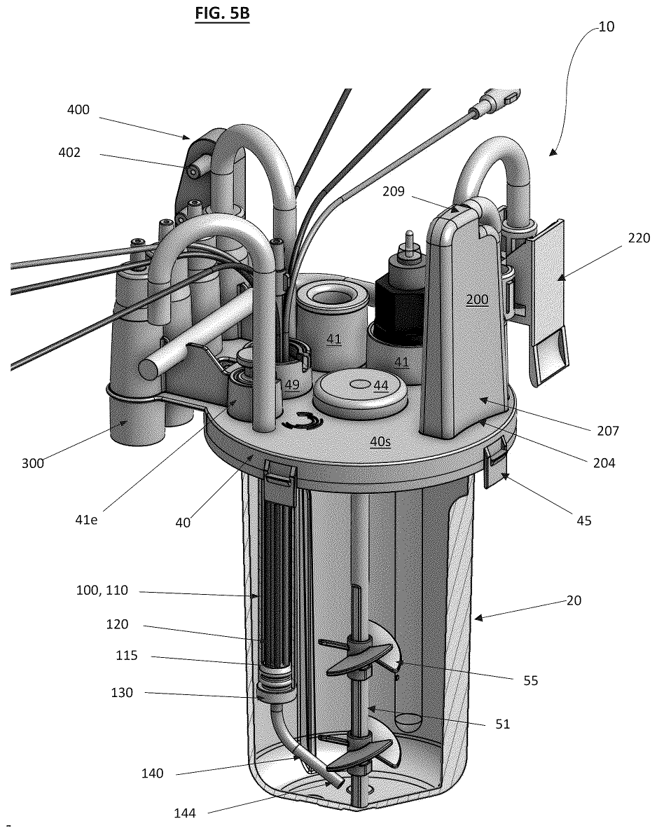

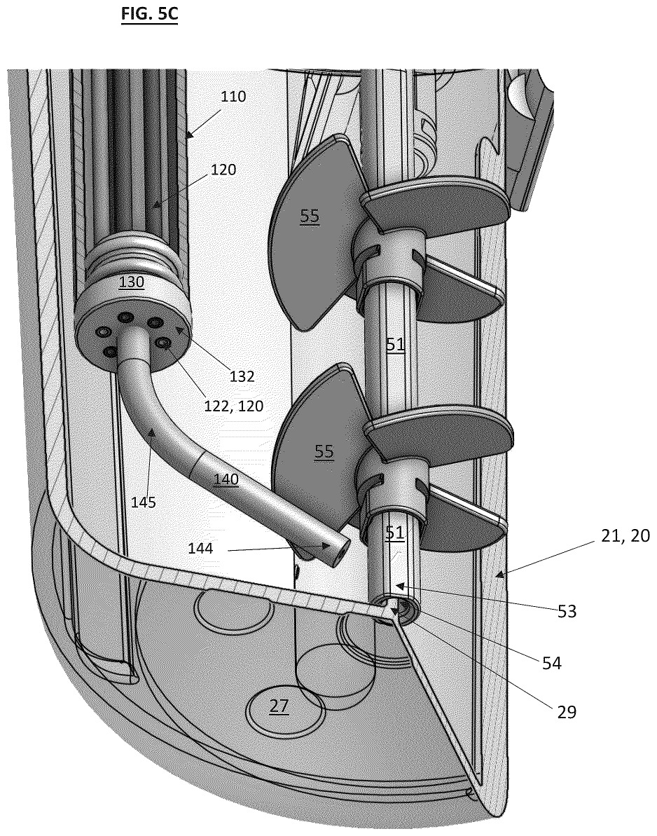

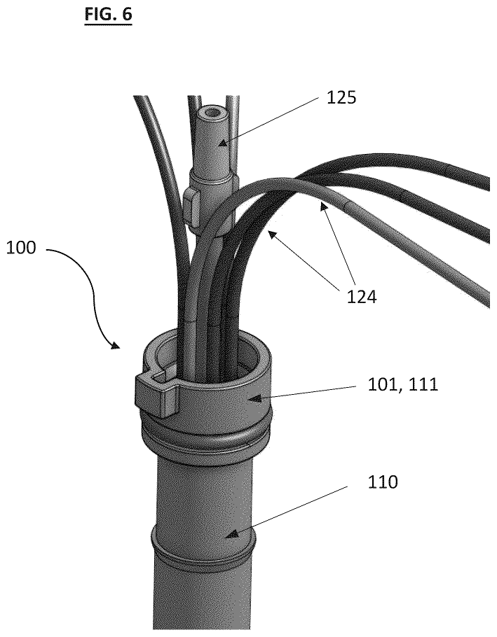

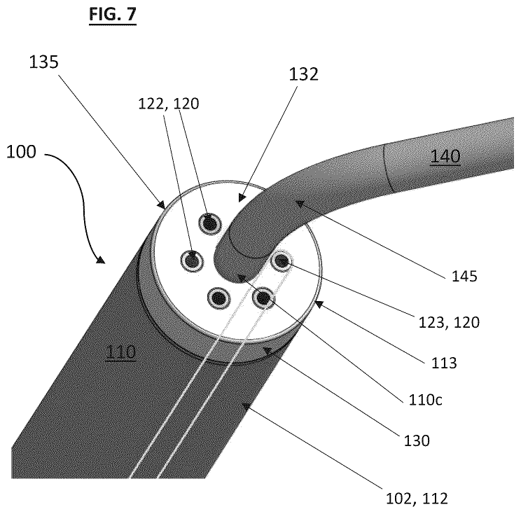

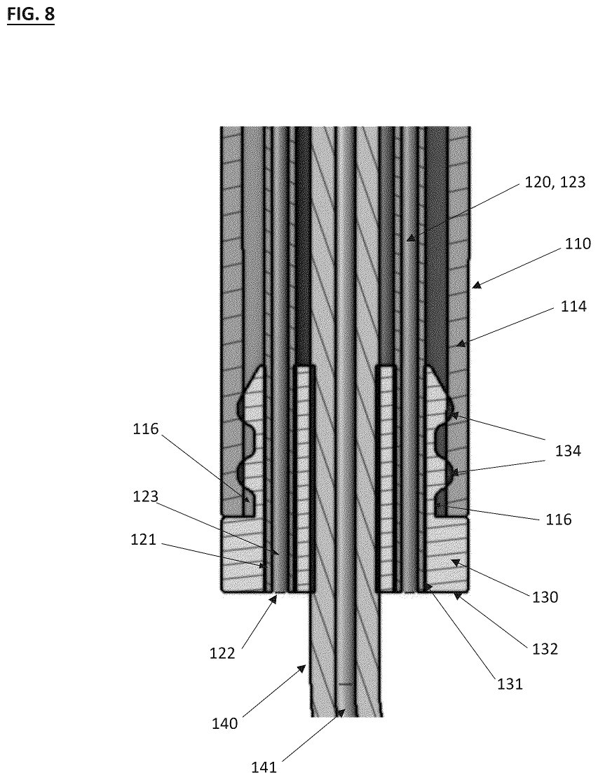

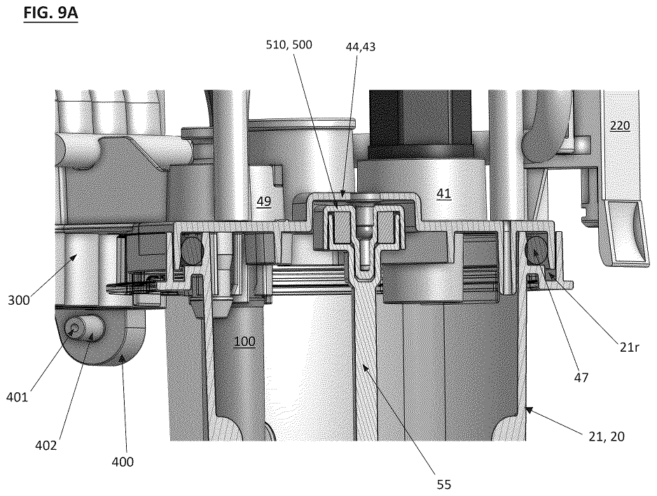

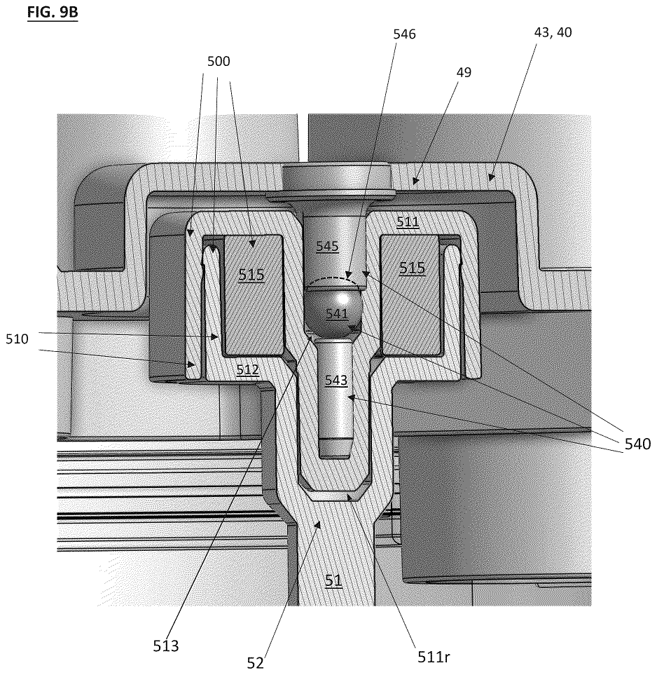

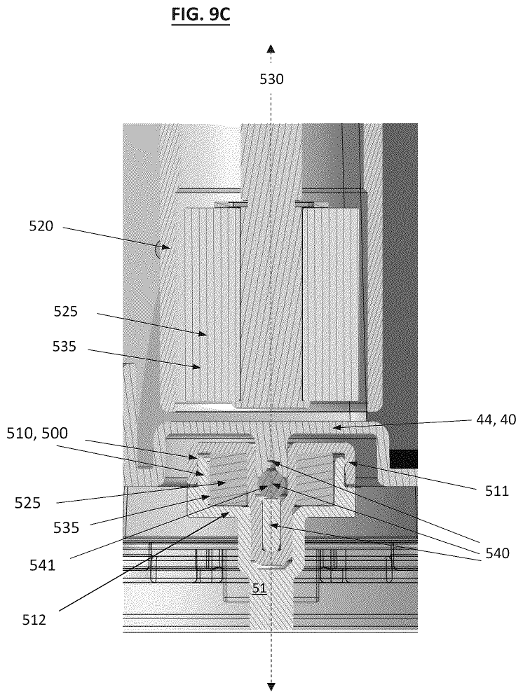

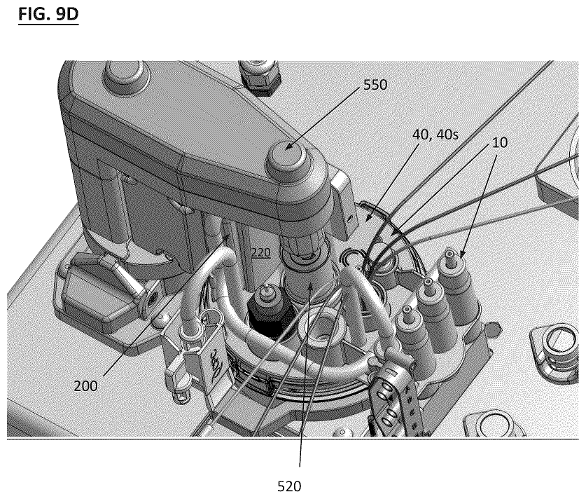

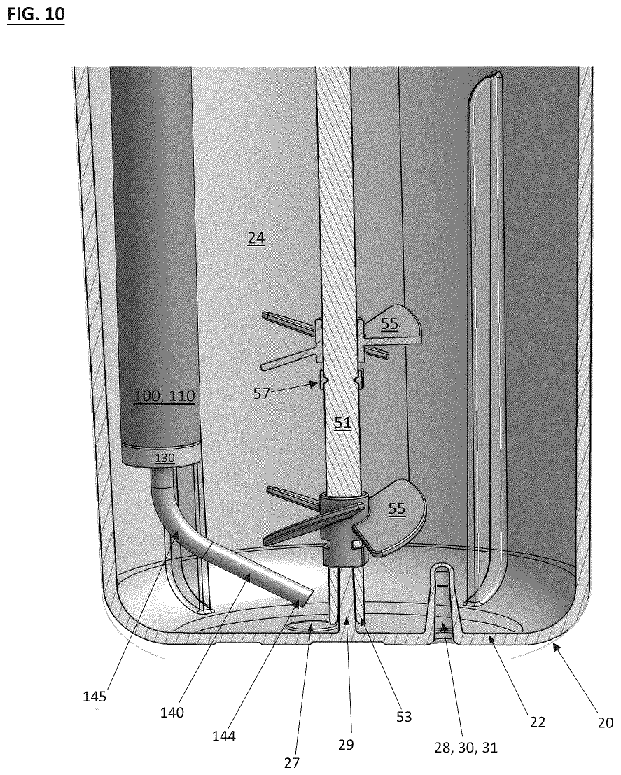

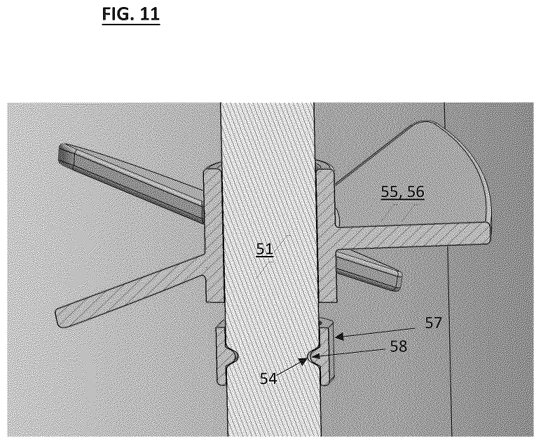

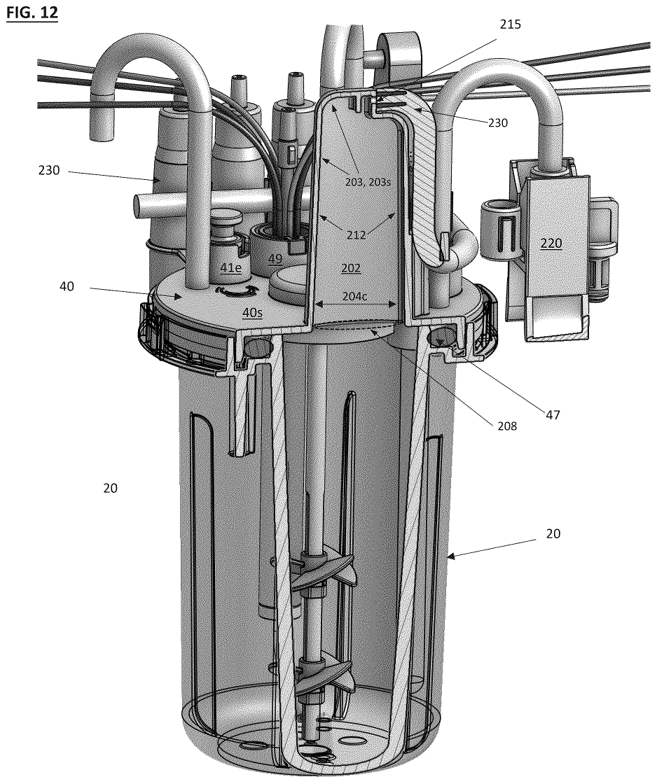

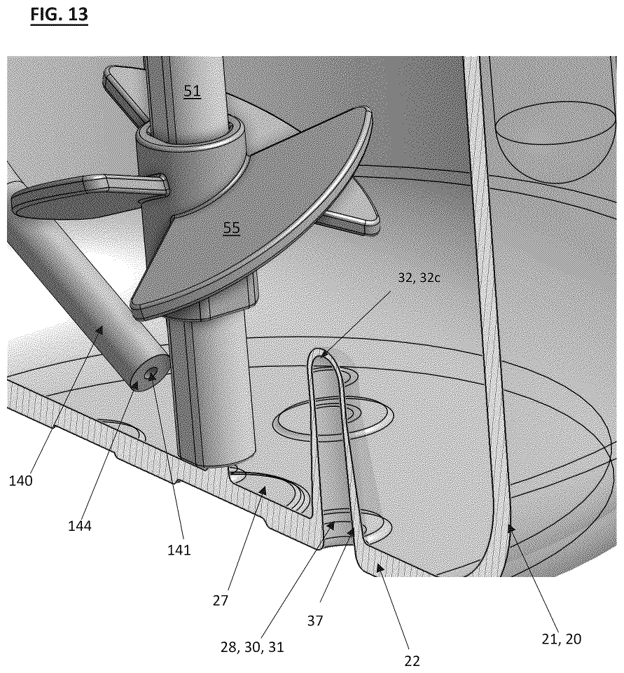

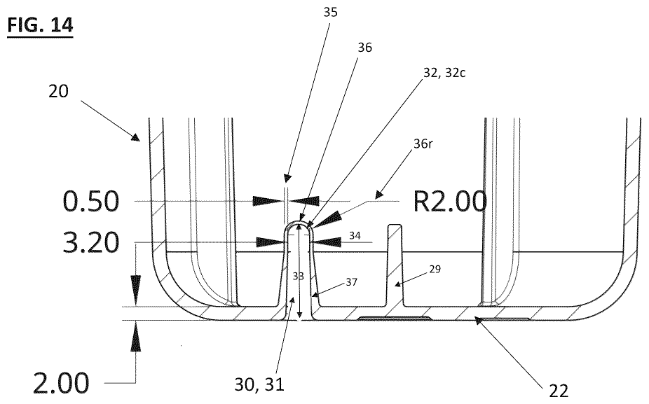

Referring now to FIGS. 1-14 an embodiment of a bioreactor 10 for incubation and growth of cells or other microorganism comprises a vessel 20, a headplate 40, an agitation system 50 including shaft 51 and impellers 55, a dip tube assembly 100, a condenser 200 with off gas conduit 230, gas manifold 300 with gas conduits 310 and multiport fluid manifold 400.

### Embodiments of the Bioreactor Vessel

Vessel 20 includes side walls 21 and a bottom wall 22 defining an interior volume or enclosure 23 for containment of liquid contents 24 such as culture media 25. In various embodiments, vessel 20 can have cylindrical-like shape with a curved bottom portion 20b. The vessel height 20h and diameter 20d can be selectable depending upon the volume of enclosure 23 (e.g. 500 ml). In particular embodiments of an approximately 550 ml volume vessel the height can range from 128 to 130 mm (with a specific embodiment of 129 ml) and the diameter 20d can range from 70.6 to 76.1 mm. In particular embodiments, the vessel side walls 21 can flare out such that the top diameter 20td of the vessel (i.e. the diameter at or near head plate 40) is larger than bottom diameter 20bd (i.e. the diameter at or near bottom portion 20b). In particular embodiments, bottom diameter 20bd can be about 70.6 mm and top diameter 20td can be about 76.1 mm. Vessel 20 can be fabricated from various polymer materials known in the art including for example rigid polycarbonate-based plastics for a relatively small volume (e.g., around 500 to 1000 ml) and may be constructed from flexible low-density polyethylene-based plastics for a relatively greater volume. In some embodiments, vessel 20 may also be configured to be re-usable and as such can be constructed from polymer materials which can be steam or radiation sterilized (e.g., via gamma radiation or e-beam).

In various embodiments vessel 20 can be fabricated from injection molding methods known in the art and can have customized size and shape and design features including one or more baffles 26 and internal recesses 27 for positioning of a patch sensor and external recesses or cavities 28 including thermal wells 30 (described in more detail herein) for positioning of temperature probes. In some embodiments, vessel 20 may be fabricated using 3-D printing methods known in the art to allow for precise customization of one or more vessel features. Also in various embodiments, all or a portion of vessel 20 can be fabricated using materials and methods so as to be transparent to allow an operator to look through the vessel. In variations, vessel 20 may also include one or more viewing windows (not shown) positioned at selection locations to allow an operator to look at selected locations in the vessel.

In particular embodiments, vessel 20 may comprise multiple baffles 26 configured to extend adjacent to vessel side wall 21 in a longitudinal direction. The baffle may have a shape that extends radially inward from the side wall and in amount selected to affect fluid flow in enclosure 23 during mixing of a culture media by one or more impeller 55 or other agitation means 55. The baffles may provide additional mounting points for sensors, probes and other actuators such as heating and cooling elements. Also in some embodiments, the baffles may be configured as actuators themselves, capable of adjusting their profile, length and number in response to dynamically changing mixing and aeration rate control profiles within the vessel.

In some embodiments, one or more of the shape, number and geometries of baffles 26 along with the arrangement of multiple baffles relative to one another or relative to the vessel may be configured to obtain a selectable flow pattern and/or mixing profile of culture media within the vessel. In some embodiments, baffles 26 can be evenly distributed around the circumference of enclosure 23 and in alternative embodiments can be variably distributed. In particular embodiments, vessel 20 may include six baffles evenly distributed around the circumference of enclosure 23.

### Embodiments of the Headplate

Headplate 40 is coupled to the top of vessel 20 (which in various embodiments can be a removable or fixed coupling) and includes one or more ports 41 for providing access to enclosure 23 by one or more or of dip tube assembly 100, gas conduits 310, probes and/or sensors and other components. In some embodiments, ports 41 may comprise expansion ports 41e and can include a removable cover 42 as is described below. Headplate 40 can also include other features 43 integral or coupled to the headplate including one or more of raised or raised portions 44 (e.g., for the magnetic drive assembly housing) condenser 200, gas manifold 300 and a drip feature. Features 43 may also comprise fittings for coupling to one or more of the aforementioned components as well as various fittings for connection of liquid and gas tubing and conduit.

In various embodiments, the one or more ports 41 in head plate 40 may include an expansion port 41e can be configured and used to add an additional probe or related component such as redundant glass pH probe, redundant oxygen probe, or other standard-sized threaded probe such as those for cell density, Raman spectroscopy, glucose/lactate, or other Process Analytical Technology probes. Typically, the expansion port 41e will include a removable cover 42 allowing a user to easily open the port as needed to add the additional component.

In still other embodiments, the headplate 40 includes a drip feature (not shown) which may be molded into the headplate or positioned in one of the ports for drip delivery of fluids such as an antifoaming agent to the culture media 24 or other liquid contents of 25 of vessel 20. In particular embodiments, the drip feature is positioned at the center of the headplate so as to have drops delivered to the center of the vessel.

In embodiments, headplate 40 is removably coupled to vessel 20 for example, by means of latches 45 on either of headplate 40 or vessel 20 or a threaded connection. In particular embodiments, headplate 40 includes a lip 46 which fits into a recess 21r at the top of vessel wall 21 with an O-ring 47 also positioned in the recess to provide for a seal between the headplate and vessel when the headplate is latched or otherwise attached into place on vessel 20 (e.g., by a threaded connection or press fit).

Headplate 40 can be fabricated from various polymeric materials known in the art including one of polymeric materials, vinyl (such as polyvinyl chloride), Nylon (such as vestamid, grilamid), pellethane, polyethylene, polypropylene, polycarbonate, polyester, silicon elastomer, acetate and so forth. Given different use applications such as disposable or re-usable, as well as sterilization methods and fermentation conditions and the like, the materials may be selected such that the materials may be substantively not corrosive, may be capable of tolerating high pressure, may be able to resist pH changes, may be able to tolerate sterilization via the application of steam, irradiation or gas, and/or may be free of toxins or materials that may react to a component or substrate from the fermentation process.

For polymeric embodiments headplate 40 can be fabricated using various molding including injection molding methods known in the polymer processing arts. In other embodiments headplate 40 may be fabricated using 3-D printing methods known in the art to allow for precise customization of one or more features of the headplate. Also in various embodiments, all or a portion of headplate 40 is fabricated using materials and methods so as to be transparent to allow an operator to look down through the headplate. In variations the headplate may also include one or more viewing windows (not shown).

### Embodiments of the Dip Tube Assembly

In many embodiments, bioreactor 10 includes a dip tube assembly 100 (also referred to as DTA 100) accordingly a description of various embodiments of DTA 100 will now be presented. DTA assembly 100 is typically positioned within one of the ports 41 of headplate 40 such that top portion 101 of the DTA extends above and out of the headplate 40 and the mid to bottom portion 102 of the DTA extends into the vessel enclosure 23. In many embodiments, the headplate 40 will include a customized port 48 for the DTA with a raised portion 49 that fits around and supports and/or stabilizes the DTA when positioned in bioreactor 10 and headplate 40.

DTA 100 typically comprises an outer tube 110 having a top; portion 111, a bottom portion 112, a bottom end 113 and a side wall 114 defining an interior volume 115. A plurality of inner tubes 120 for delivery and/or sampling of liquids and gases are disposed within the interior volume 115 of the outer tube 110. Each inner tube 120 includes a top and bottom end 121 and 122 and lumens 123 for the passages of liquids and gases. The top end 121 of one or more inners tube 120 will typically be coupled (or configured to be coupled) to a tubing segment 124 having a connector 125 for fluidically coupling inner tubes 120 to one or more of sources of liquids and gasses or to sampling devices or containers (not shown). In particular embodiments, tubing segments 124 including connectors 125 can be coupled to one or more ports 402 of multiport fluid manifold 400 for the delivery of fluid via a fluid delivery device (e.g. a syringe or syringe pump) fluidically coupled ports 402 of manifold 400. In one or more embodiments, the DTA 100 can include between four to ten inner tubes 120 which may be evenly radially distributed within the interior volume 115 or in some instance one inner tube 120 will be positioned at a center 110c of the outer tube and the other inner tubes distributed around it. In particular embodiments, DTA 110 will include seven inner tubes, one which is positioned at center 110c of the outer tube and the other six radially distributed around the center. The center positioned inner tube 120 will typically be used for delivery of sparging gas.

A plug 130 is positioned within a bottom portion of the interior volume 115 of the outer tube 110 and will typically have a rounded shape to fit into the interior volume of the outer tube 100. The plug 130 includes a plurality of lumens 131 in/through which the plurality of inner tubes is positioned. In some embodiments, the bottom ends 122 of the inner tubes 120 extend out of bottom surface 132 of the plug 130 by a selected amount for example, in a range of about 1 to 5 mm or 2 to 3 mm. In other embodiments, the bottom ends 122 of the inner tube 120 are substantially flush with bottom surface 132 of plug 130.

The shape, material properties and other features of the plug 130 are desirably configured to form a fluidic seal around each inner tube 120 such that fluid contents of the bioreactor 10 do not enter the interior volume 115 of the outer tube 110 when dip tube assembly 100 is positioned within the bioreactor. The plug also includes at least one protrusion 134 positioned on a side surface of the plug 133. Protrusion(s) 134 is sized to form an interference fit and fluidic seal between plug 130 and an interior surface 116 of the outer tube 110 such that liquid contents of the bioreactor do not enter the interior volume 115 of the outer tube when the dip tube assembly is positioned within the bioreactor. Typically, plug 130 will include two such protrusions 134 and they will extend around the entire circumference 135 of the plug. Also, in one or more embodiments a potting agent (not shown) is injected or otherwise disposed in outer tube interior volume 115 at or around the inner top surface 135 of plug 130 and around inner tube 120 to provide for additional sealing and water tight ability of the seal formed between plug 130, outer tube 110 and plug 130 and inner tubes 120.

In one or more embodiments, plug 130 is configured to have a coefficient of thermal expansion matched to a coefficient of thermal expansion of the outer tube 130 such that the fluidic seal between plug 130 and the outer tube 120 is maintained upon heating of the outer tube by culture media 25 or other liquid contents 24 of bioreactor 10. Matching of the respective coefficients of thermal expansion can be achieved by selection of the materials and fabrication methods for the plug and outer tube.

In many embodiments, DTA 100 will also include a down tube 140 for the delivery of sparging gas to a selected location in the bioreactor vessel 20. Downtube 140 (also sometimes referred to herein as a downpipe sparger) is coupled to a bottom portion/surface 132 of the plug and includes an inner lumen 141 that is fluidically coupled to a bottom end 122 of one of the inner tubes 120. Typically, down tube 140 which will be coupled to a center portion of the plug bottom surface 132 and as such will be fluidically coupled to be a center positioned inner tube 120 as described above. However other locations for positioning of the down tube 140 on the plug surface 132 are also contemplated. In many embodiments, down tube 140 will include an elbow portion 145 that is shaped or otherwise configured to direct a bottom end 144 of the down tube (including the down tube inner lumen) near or towards a high mixing zone of an agitation impeller 55 or other agitation element coupled to agitation shaft 51 within bioreactor vessel 20. In some embodiments, down tube 140 and elbow portion 145 can be configured (e.g., sized and shaped) to locate the down tube end within about 5 to 20 mm from an agitation impeller 55. In particular embodiments, the down tube 140 and elbow portion 145 can be configured to position the down tube end 144 at a location between impeller and the bottom of the bioreactor vessel (e.g., equidistant between the two). In various embodiments, the exact location of the down tube end 144 can be selected depending upon one or more of the volume of liquid in the vessel, the rotational velocity of the agitation shaft and the flow rate of the sparging gas as well as the desired dissolved gas concentration (e.g., O2) to be obtained within the culture media or other vessel liquid. In some embodiments selection of the down tube end position can be achieved by embodiments of the downtube 140 which are configured to be telescoping and/or through the use of a set of detachable down tubes of various lengths.

In use, embodiments of the bioreactor having a dip tube assembly 100 including those with a down tube 140 provide the advantage of aiding to repeatably position all the gas/liquid addition and sampling ports at a defined location and in the gas of sparging gas near to the agitation impeller(s) to ensure fast mixing and in turn reproducibility of results across reactors.

### Embodiment of the Condenser

In many embodiments bioreactor 10 includes a condenser 200 for condensing liquid from gas flowing out of bioreactor 10 and enclosed vessel 20. Accordingly, a description of embodiments of condenser 200 will now be provided. Condenser 200 will typically be positioned on headplate 40 either by being formed integrally with headplate 40 or by being attached to it by adhesive or a mechanical joint. Condenser 200 has a vertical oblong shape 201 rising above the headplate defining an interior volume 202 (having an interior shape 203), an oblong horizontal shape 204 including two long sides 205 and two short sides 206 and a bottom portion and a top portion 207 and 209. The bottom portion 207 has an oblong opening 208 continuous with headplate 40. The top portion 209 includes an opening 215 positioned at a short side 206 for the outflow of gas. At least one of the long sides 205 is configured to be thermally coupled with a movable chilling structure 220 such as rectangular chilling block which may be rectangular shaped. Also, at least one of the interior condenser shape 203 or horizontal dimensions along a vertical axis 211 of the condenser 200 are configured to minimize blockage of the interior volume 202 by liquid or foam bridging or otherwise spanning across interior walls of the condenser.

The horizontal profile or cross-section 204c of condenser 200 including the bottom portion and bottom opening 207 and 208 along with the top portion 209 will typically have an oval or other oblong asymmetric shape sized configured to inhibit or reduce liquid or foam blocking the condenser bridging or otherwise spanning across the interior walls 212 of the condenser. Accordingly, in these and related embodiments, the long sides 205 will be flat and the short sides 206 curved. Condenser 206 will also typically have a decreasing vertical taper to facilitate condensation of water vapor and provide for ease of outflow gas connection to the condenser.

In one or more embodiments, at least one of the shape or dimensions of condenser 200 are configured to minimize loss of vessel liquid contents resulting from condensed liquid escaping out of the gas outflow opening 215 and/or inefficient condensation of gas flowing through the condenser. Accordingly, in these and related embodiments, the condenser long sides 205 can have a length in a range from about 20 to 30 mms, the short sides 206 a length in a range from about 5 to 10 mm and the height 210 of the condenser can range from about 50 to 70 mm. In these and related embodiments, the liquid loss can be less than about 5 per cent of the vessel liquid contents per day of operation of the bioreactor and more preferably about one per cent per day of operation of the bioreactor. Also in these and related embodiments, condenser shape 201 and its dimensions provide for sufficient internal surface area 203s to condense at least about 90 percent of the water vapor flow through the condenser. In various embodiments the internal surface area of the condenser can range from about 15,710 mm to about 65,973 mm.

Various embodiments of condenser 200 can also include other features and aspects to reduce or prevent blockage of the interior volume 202 of the condenser by water droplets and/or foam. For example, in some embodiments, an interior surface 212 of the condenser has a hydrophobic surface tension configured to minimize condensed liquid from adhering to the interior surface. In particular embodiments, the interior surface tension is configured to induce condensed liquid to fall or roll down the condenser interior surface 212. In one or more embodiments, the surface tension of the condenser can be below about 50 dynes/cm, more preferably below 40 dynes/cm and still more preferably below about 30 dynes/cm.

Various embodiments of the condenser can also be configured to enhance cooling of the condenser by chilling structure 220 and thus condensation of water vapor flowing through the condenser. For example, in one or more embodiments, the long side(s) 205t for thermal coupling to the chilling structure can include an additive or coating 205c for enhanced thermal conductivity. Such coatings or additives can include thermally conductive polymers or metals known in the art. In these and related embodiments including thermally conductive coatings or additives, the thermal conductivity of the thermally coupled long side can be at least about 1 W/(m K) or greater and more preferably at least about 10 W/(m K) Additionally in one or more embodiments, the condenser structure 213 including the long side(s) for thermal coupling 205t can be configured to be put under compressive loading or force by the chilling structure (when it is moved into place against the long side e.g., by movement of the gantry) so as to enhance conduction and heat flux between from the condenser structure including the long side to the chilling structure. In these and related embodiments, the surface contour of the long side can substantially match or correspond to that of the chilling structure. For example, for embodiments of a rectangular shaped chilling structure 220, the contour 205tc of the thermally coupled long side 205t can be substantially flat. Similarly, for embodiments where the chilling structure has a curved shape the contour 205tc of the coupled long side can have a matching curved shape.

### Embodiment of the Thermal Well

In various embodiments, vessel 20 includes a thermal well 30 a thermal well for improved temperature measurement of the liquid contents of the vessel by an inserted temperature probe. Accordingly, a description of thermal well 30 will now be provided. Thermal well 30 will typically comprise an upwardly extending cavity 31 formed within vessel bottom 22. Desirably, the cavity shape 32, height 33, width 34, and wall thickness 35 are configured to allow for greater than a 99 to 99.5 per cent or even greater accuracy in a temperature measurement of liquid contents surrounding the cavity by the inserted thermal probe. Typically, the cavity will have an upward cylindrical shape 32c with a curved end 36 and also may have a decreasing vertical taper. Curved end 36 can have a selected radius of curvature 36r.

In one or more embodiments, the dimensions of well 30, cavity 31 can be configured for a slightly snug fit around the temperature probe such that surface of the temperature probe makes complete or near complete contact with cavity surface 37 (i.e., the surface of the vessel bottom wall defining the cavity) for enhanced thermal conduction the cavity wall to the temperature probe. Accordingly, in these and related embodiments the width 34 and length 33 of the cavity can be selected to match those of various temperature probes known in the art including specific temperature probes used for measurement of bioreactor temperature. In particular embodiments, the cavity has a length 33 between about 13 to 15 mms a width 34 of between about 3 and 3.4 mm and a wall thickness 34 between about 0.4 mm and 0.6 mm with a specific embodiment of about 14 mm height, a 3.2 mm width and a 0.5 mm wall thickness. The radius of curvature 35r of cavity end 35 can range from about 1.5 to 2.5 mm with a specific embodiment of 2.0 mm. As indicated in the Appendix, an embodiment of the thermowell having the above specifically defined mentions yielded a modeled temperature accuracy of greater than 99.65 per cent.

In addition to dimensions, one or more embodiments of thermal well 30 may include other means for improving accuracy of temperature measurements by the inserted temperature probe. For example, similar to the thermally coupled condenser long side, in some embodiments, the outer walls of the thermal well 30/cavity 31 may include a coating or additive for enhanced thermal conductivity.

### Embodiments of the Gas Manifold

In some embodiments, bioreactor 10 will also include a multiport gas manifold (MPGM) 300 a description of which will now be provided. MPGM) 300 includes a plurality of separate gas channels 301 each having an inlet and outlet 302 and 303 and conduit 310 extending from the channel outlet. Typically, channels 302 will have a tapered shaped 302t with the inlet 302 being larger than the outlet 303. At least one of channels 301 is fluidically coupled to the dip tube assembly 100 for the delivery of a sparging gas through the assembly. One of channels 301 can also be coupled to headplate 40 for the delivery of gas (known as overhead gas) to the space in vessel 20 above the liquid contents 24 (e.g., the culture media). In particular embodiments, MPGM 300 includes four gas channels 301, two coupled to the dip tube assembly 100 for the delivery of sparging gas (e.g., O2, CO2, Nitrogen, etc.), one coupled to the headplate 40 for the delivery of overhead gas and one for gas flowing out of vessel 20 described herein as off-gas which will typically go through an embodiment of condenser 200 described herein.

In various embodiments, MPGM 300 can have various features and attributes to facilitate connections at the channel inlet and outlet, control gas flow rates and maintain sterility of gases flowing into the bioreactor. For example, in some embodiments, channel inlets 302 (or other portions of the channels) may include or be configured to be coupled to one or more mass flow controllers (not shown) so as to control the flow of gas into each channel 301.

Also in some embodiments, channels of MPGM 300 may contain or be configured to contain sterile filters 320 for filtering out microbes (e.g., bacteria, fungi and viruses) and particulates. The filters 320 may be a standard shape or custom fitted for the shape of gas channels 301. Typically, they will be press-fit into the gas channel. In particular embodiments, filters 320 may have a pore size of 0.2 μm or less. Also, in some embodiments the channel outlets 303 can include an extended fitting to allow for insertion and secure connection of tubing or other conduit connecting the outlets to the DTA or headplate. Also, the channel inlets 302 can include or be configured to be coupled with rigid O-rings seals (e.g., fabricated from high durometer silicone) which serve to ensure that reliable connections are made to all of the gas channel inlets 302.

### Embodiments of the Multi-port Fluid Manifold

In one or more embodiments, headplate 40 or other portion of bioreactor 10 can be coupled or is configured to be coupled to a multi-port fluid (e.g. liquid) manifold 400 (MPFM) including a plurality of separate connection ports 402 and corresponding channels for fluidically coupling at least a portion of the DTA inner tubes 120 to separate sources of liquid, such as liquid filled syringes. Typically, the MPFM 400 will include five ports 402 and corresponding fluid channels/lumens 401, but other numbers are also contemplated. As discussed above, in many embodiments, the ports 402 of MPFM 400 can be coupled to external tubing segments (not shown) which can be supplied with bioreactor 10 and configured to be coupled to syringes or pumping/fluid source means which are used to deliver fluid to DTA 100 and/or headplate 40.

### Embodiments of the Agitator and Magnetic Drive System

The agitation assembly 50 will typically include drive assembly 500 (such as magnetic drive assembly 500 described below) operatively coupled to a draft shaft 51 coupled to an impeller or other agitation means 55. In some embodiments impeller 55 is fixedly coupled to shaft 51. However, in additional or alternative embodiments, impeller 55 can be detachably coupled to shaft 51 such that the impeller can be positioned at selected locations along the length of shaft 51. In particular embodiments, this can be achieved by clip on stops 57 which can be positioned below and/or above impeller 55. Stops 57 typically include protrusions 58 which snap or otherwise fit into and engage recesses 54 so as to lock into shaft 51.

In many embodiments, bioreactor 10 includes an agitation assembly 50 that includes a magnetic drive assembly 500 a description of which will now be provided. In one or more embodiments, magnetic drive assembly 500 includes a protective housing 510 and a first diametric magnetic 515 positioned in the housing that is configured to be magnetically coupled to a second diametric magnetic 525 positioned above headplate 40 in a rotating housing 520 so as to rotatably drive agitation shaft 51 by rotation of second magnet 525. The second diametric magnet 525 rotatably drives the first magnet 515 by magnetic lines of force substantially orthogonal to an axis of rotation 530 of the two magnets. In many embodiments, the first and second diametric magnets 515 and 525 have a toroidal shape 535 such as a square, rectangular or circular toroid; however other shapes for the diametric magnets are also contemplated.

In various embodiments, the drive assembly housing 510 comprises a first part 511 and a second part 512 which is fixedly inserted into first part 511 to define an interior space 513 containing the first magnet 515 and form a substantially watertight seal around the interior space 513 and magnet 515. The second part 512 is integral or otherwise fixedly attached to a proximal end 52 of agitation shaft 51. In particular embodiments, first and second housing parts 511 and 512 can be configured for the second part to have a snap fit into the first part using protrusions and/or detent features in one or both parts.

In many embodiments, the magnetic drive system housing 510 also includes a bearing system 540 for reducing friction during rotation of the agitation shaft. In particular embodiments, the bearing system 540 can be at least partially positioned or contained in a recess 512r formed in first housing part 512. According to various embodiments, bearing system 540 comprises a bearing 541, a first bearing contact structure 543 positioned below the bearing and a second bearing contact structure 545 positioned above the bearing. The first bearing structure 543 will typically comprise an elongated stainless steel dowel pin or other metal pin that is fixedly inserted into the recess. Such elongated metal dowel pins or other like structures provide the benefit of conducting heat away from the bearing surface, reducing wear of the bearing. The second bearing contact structure 545 may comprise a post or other structure that is fixedly inserted into a bottom surface 49 of headplate 40, typically at the center of the headplate. Bearing 541 will typically correspond to a ball bearing but configurations using roller bearing or even magnetic bearings are also contemplated. For embodiments of the bearing system using ball bearings, the bearing may comprise one or more wear resistant materials known in the art including for example various wear resistant polymer such as polyamide-imide with a specific example being TORLON. Also, for embodiments where the bearing is a ball bearing, the post or other second bearing contact structure 545 can have a cup-shaped contact surface 546 configured to center the bearing. In specific embodiments, the radius of curvature of the cup surface 546 can correspond to that of the ball bearing so as to optimize the fit between the bearing and cup surface.

In such embodiments of the magnetic drive and bearing systems 500 and 540 as described above, the systems are configured to operate such that during rotational movement induced by second magnet 525, the first magnet 515 rotates (along with the agitator shaft and the dowel pin) while ball bearing 541 remains stationary, forming the wear surface at the intersection of the dowel pin and ball bearing. The metal dowel pin 543 also provides an additional function and benefit of drawing heat down and away from the wear surface 542 of bearing 541 to increase lifetime of the bearing system and prevent overheating.

In various embodiments, the second diametric magnet 525 will typically be positioned in a rotatable housing 520 positioned in proximity to the headplate outer surface 40s above the drive system housing 510 such the two diametric magnetic 515 and 525 are substantially axially aligned. In many embodiments, the rotatable housing 520 is positioned or otherwise coupled to a movable gantry 550 configured to move the rotatable housing and second magnet in axial alignment with the drive housing and first magnetic so as to magnetically couple the two magnets.

In some embodiments of magnetic drive assembly 500, the bioreactor headplate 40 can include a raised portion 44 and the magnetic drive assembly housing 510 can be at least partially positioned within the raised portion of the headplate. Such embodiments provide the advantage of reducing the space requirements for the drive system housing and position the housing away from the liquid in the vessel reducing the likelihood of vessel liquid contents from getting on or into the housing. They also facilitate positioning, alignment and magnetic coupling of the rotatable housing/second magnetic with the drive housing/first magnet as owing to the height of the raised portion above the headplate surface (which can be in the range of about 10 to 30 mm), the two housings can be brought into close proximity without interference by other components of headplate 40.

### Definitions

The terms “substantially” and “about” are used herein to describe and account for small variations including small variations in a recited, parameter, property, quality, or dimension. For example, when used in conjunction with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

As used herein, the singular terms “a,” “an,” and “the” may include plural referents unless the context clearly dictates otherwise. Also, reference to an object in the singular is not intended to mean “one and only one” unless explicitly so stated, but rather “one or more.

Additionally, amounts, ratios, and other numerical values may sometimes be presented herein in a range format. As used herein, a range of numbers includes any number within the range, or any sub-range if the minimum and maximum numbers in the sub-range fall within the range. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. Thus, for example, “<9” can refer to any number less than nine, or any sub-range of numbers where the minimum of the sub-range is greater than or equal to zero and the maximum of the sub-range is less than nine. Ratios may also be presented herein in a range format. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

### Appendices/Examples

Various embodiments are further illustrated with reference to the following appendices/examples. It should be appreciated that these examples are presented for purposes of illustration only and that the subject matter is not to be limited to the information or the details therein.

### Appendix 1: Thermal Modeling of the Bioreactor Thermal Well

As described above, various embodiments of the bioreactor include a thermowell. The thermowell comprises a small protrusion in the bottom wall of the bioreactor vessel that is configured to allow the tip of a temperature probe to be inserted and surrounded on three sides by portions of vessel wall which are themselves in contact with fluid in the vessel thereby measuring temperature of the fluid. Initial designs of the thermowell resulted in thermowell measured temperatures of vessel fluid being about 3° C. cooler than the actual temperature of fluid in the vessel (as measured by a secondary temperature probe inserted from the top of the bioreactor and immersed in the vessel fluid). It was speculated that this temperature difference was due to one or more of the vessel wall thickness, thermal resistance of the vessel polycarbonate material and cooling effects from the ambient air around the outside of the vessel. With these in mind design changes were made in order to improve the accuracy of the thermowell measured vessel temperature. The design changes were three-fold and intended to improve heat transfer from the vessel fluid to the temperature probe while reducing potential cooling effect from ambient air around the vessel. First, the depth of the thermowell was increased as much as possible without resulting in a collision with other internal components inside the vessel. Second, the tolerance fit between the probe tip and the thermowell was tightened (i.e. reduced) to increase the contact surface area and reduce air gaps. Third, the vessel wall thickness at the tip of the thermowell was reduced by about half, the most that was estimated could be thinned without introducing molding errors such as insufficient flow into the feature. The specific design changes were as follows: i) the depth of thermowell was increased from 8 to 12.5 mm; ii) the wall thickness near the tip or top of the well was reduced from 1 mm to 0.5 mm; and iii) the tolerance (i.e., gap) between the probe sides and the vessel surface was reduced from 0.22 mm to 0.02 mm (an approximation, as draft angles are involved). In order to gain an estimate of the effects of these design changes before they were made the thermal equilibrium of the vessel, vessel holder, probe, and probe holder were modeled before and after the geometry changes. The software used for all thermal modeling was the SimSolid modeling plugin for OnShape CAD. The model assumptions were as follows. First, the inside surface of the vessel was held constant at 37° C. (which is a common cell culture temperature). Next, the “contacts” where direct thermal conduction could occur were defined as surfaces 0.05 mm apart or less. Also, the outer surfaces of the vessel holder, temperature probe, and probe holder were allowed to experience convection to a room temperature air environment (defined as 20° C.) without wind. Specific materials were assigned to each modeled solid (e.g., polycarbonate, polypropylene, and stainless steel for the bioreactor vessel, vessel holder, and probe/holder respectively). The thermal modeling software then used these material assignments to automatically apply heat transfer coefficients (e.g. thermal conductivity coefficients).

Using the above assumptions, the original geometry of the thermowell was thermally modeled, and found to be in good agreement with measured observations, as the top ˜5 mm of probe tip, when averaged, yielded a temperature of approximately 34° C. Holding the assumptions and conditions the same, the new geometry (i.e., that having the selected new dimensions described above) of the thermowell was substituted in and the model repeated. The results showed that in similar conditions, the averaged measured temperature for the top ˜5 mm portion of the probe was 36.873° C. which corresponds to an accuracy of greater than 99.65 per cent. Thus, the error in temperature measurement of vessel liquid by an inserted temperature probe was reduced from ˜3° C. to less than 0.13° C., a more than 23-fold improvement. This degree of improvement was surprisingly larger than anticipated.

While various embodiments have been shown and described herein, it will be obvious to those skilled in the art that such embodiments are provided by way of example only. Numerous variations, changes, and substitutions will be apparent to those skilled in the art without departing from the disclosure. For example, embodiments of the bioreactor and respective sub-components described herein can be adapted for use with a variety of cell types including mammalian, bacterial and yeast cells. They can also be adapted for the growth and/or of cells for a variety of applications including one or more of production of antibodies and other biopharmaceuticals; and one or more of the enrichment, expansion and production of populations of cells for cell therapies (e.g., T-cells, CAR-T-cells, other CAR immune cells and the like) and production of viruses and viral vectors for gene therapy and other applications.

Elements, characteristics, or acts from one embodiment can be readily recombined or substituted with one or more elements, characteristics or acts from other embodiments to form numerous additional embodiments within the scope. Moreover, elements that are shown or described as being combined with other elements, characteristics, steps or acts can, in various embodiments, exist as stand-alone elements, characteristics, steps or acts. Further, various embodiments expressly contemplate the negative recitation of any element, characteristic, step or act etc. that is/are shown or described in one or more embodiments. Hence, the scope is not limited to the specifics of the described embodiments but is instead limited solely by the appended claims.

All references, including publications, patent applications, and patents, cited herein are hereby incorporated by reference to the same extent as if each reference were individually and specifically indicated to be incorporated by reference and/or were set forth in its entirety herein.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

### Incorporation by Reference

All publications, patents, and patent applications mentioned in this specification are herein incorporated by reference to the same extent as if each individual publication, patent, or patent application was specifically and individually indicated to be incorporated by reference.

1. A dip tube assembly for use with a bioreactor, the dip tube assembly comprising;
an outer tube having a top end, a bottom end and a side wall defining an interior volume;
a plurality of inner tubes disposed within the interior volume of the outer tube, the inner tubes having inner lumens and top and bottom ends;
a deformable plug positioned within a bottom portion of the interior volume, the plug having a plurality of lumens through which the plurality of inner tubes are positioned, the plug configured to form a fluidic seal around each inner tube of the plurality such that fluid contents of the bioreactor do not enter the interior volume of the outer tube when the dip tube assembly is positioned within the bioreactor; and
at least one protrusion positioned on a side surface of the plug, the at least one protrusion sized to form an interference fit and fluidic seal between the plug and an interior surface of the outer tube such that liquid contents of the bioreactor do not enter the interior volume of the outer tube when the dip tube assembly is positioned within the bioreactor;
wherein the plug has a coefficient of thermal expansion matched to a coefficient of thermal expansion of the outer tube such that the fluidic seal between plug and the outer tube is maintained upon heating of the outer tube by liquid contents of the bioreactor.

2. The dip tube assembly of claim 1, wherein the bottom ends of the inner tubes are substantially flush with a bottom surface of the plug.

3. The dip tube assembly of claim 1, wherein the bottom ends of the inner tubes extend out up to three millimeters from the bottom surface of the plug.

4. The dip tube assembly of claim 1, wherein the protrusions of the plug comprise at least two protrusions.

5. The dip tube assembly of claim 1, further comprising a down tube coupled to a bottom surface of the plug, the down tube configured to deliver sparging gas to fluid contents in the bioreactor and having an inner lumen fluidically coupled to a bottom end of an inner lumen of at least one of the inner tubes, the down tube having an elbow portion configured to direct a bottom end of the down tube inner lumen near or towards a high mixing zone of a bioreactor agitation impeller or other agitation element.

6. The dip tube assembly of claim 5, wherein the elbow portion is configured to direct the down tube inner lumen to a location in the bioreactor vessel between the agitation impeller and the bottom of the vessel.

7. The dip tube assembly of claim 5, wherein the down tube is positioned on a center portion of a bottom surface of the plug.

8. A bioreactor for growing cells, the bioreactor comprising:
a vessel defining an inner volume configured to contain liquid contents;
a head plate for coupling a plurality of components to the bioreactor, the head plate coupled to at a top portion of the vessel and including at least one port; and
the dip tube assembly of claim 1, wherein the dip tube assembly is positioned within at least one port such that a top portion of the dip tube extends above the headplate and a bottom end of the dip tube extends into the vessel inner volume.

9. The bioreactor of claim 8, further comprising a plurality of external tubing segments, each external tubing segment coupled to a top end of each inner tube.

10. The bioreactor of claim 8, further comprising an agitation shaft coupled to the headplate and extending downward into the vessel inner volume, the agitation shaft including at least one agitation element coupled to the agitation shaft, and
wherein the bottom end of the down tube is positioned within a high mixing zone of at least one agitation element.

11. The dip tube assembly of claim 1, wherein the elbow portion is configured to direct the down tube inner lumen to a location in the bioreactor vessel between the agitation impeller and the bottom of the vessel.

12. The bioreactor of claim 8, further comprising a multiport gas manifold coupled to or with integral with the headplate, the gas manifold including a plurality of separate gas channels each having an inlet and outlet, wherein at least one of the gas channels is fluidically coupled to at least one of the dip assembly or the headplate.

13. The bioreactor of claim 12, wherein the gas manifold includes four separate gas channels.

14. The bioreactor of claims 12, wherein the manifold includes two channels fluidically coupled to the dip tube assembly for delivery of sparging gasses, one channel fluidically coupled to the headplate for delivery of overhead gasses and one channel for off gassing of gas from the bioreactor vessel.

15. The bioreactor of claim 12, wherein a portion of each gas channel contains or is configured to contain a filter for filtering one or more of microbes and particulates.

16. The bioreactor of claim 13, wherein the filter has a pore size of 0.2 μum or less.

17. The bioreactor of claim 12, wherein the gas channel inlets include or are configured to be coupled to O-ring seals.

18. The bioreactor of claim 12, wherein the gas channel inlets include or are configured to be coupled to mass flow controllers.

19. The bioreactor of claim 12, further comprising a multi-port fluid manifold associated with the head plate, the fluid manifold including a plurality of separate channels for fluidically coupling at least a portion of the inner tubes to separate sources of liquid.

20. The bioreactor of claim 19, further comprising a plurality of external tubing segments, each external tubing segment coupled to a port on the multi-port fluid manifold.

21. The bioreactor of claim 8, wherein the headplate comprises at least two ports.

22. The bioreactor of claim 21, wherein at least one of the ports comprises an expansion port sized for the insertion of a standard threaded probe, the expansion port including a removable cover.

23. The bioreactor of claim 21, further comprising a drip element positioned in one of the ports, the drip element configured to be coupled to a fluid source.

24-71. (canceled)
